(19) United States
(12) Patent Application Publication
(10) Pub. No.: US 2018/0000425 A1
(43) Pub. Date: Jan. 4, 2018

(54) MIGRAINE HEADACHE TRIGGER DETECTION PROCESSING

(71) Applicant: Brian J. Hernacki, Santa Clara, CA (US)

(72) Inventor: Brian J. Hernacki, Santa Clara, CA (US)

(21) Appl. No.: 15/198,602

(22) Filed: Jun. 30, 2016

(51) Int. Cl. A61B 5/00; A61B 5/024

(52) U.S. Cl. CPC A61B 5/7275; A61B 5/6803; A61B 5/746; A61B 5/681; A61B 5/024; A61B 5/4824; A61B 5/4812; A61B 5/4266; A61B 2560/0242

(57) ABSTRACT

Techniques and device configurations used in the detection of migraine triggers or similar human pain conditions are disclosed. In an example, a migraine trigger detection device collects data on ambient stimuli for a human subject, through various light, sound, or odor sensors. A computing device connected to the migraine trigger detection device receives and processes the data to correlate the detected stimuli with migraine symptoms of the human subject. Such correlation may be based on real-time data, data from a prior phase of the migraine, or data from other migraine episodes (e.g., to identify common triggers of migraines over time). Additionally, such correlation may be performed with use of a remote data processing system that receives the sensor data (or aggregated forms of data). In further examples, trigger reports, pain condition, time inputs, or migraine phase may be obtained through a graphical user interface of the computing device.

### TECHNICAL FIELD

Embodiments described herein generally relate to data detection and condition processing occurring in electronic devices used by human users, and in some examples, data detection and condition processing for complex and variable medical conditions related to the occurrence of a migraine headache or like sensory-based condition in a human subject.

### BACKGROUND

A large portion of the population suffer migraine headaches (simply referred to as “migraines”) at some point in their life. Estimates vary, but common attributed numbers indicate 37 million people in the United States experience migraines, with 2-3 million of this population characterized as experiencing chronic migraines. Migraine headaches cause significant pain and result in many undesirable effects (including disorientation and inability to work).

Unfortunately, migraines are not fully understood by current medical science. Many types of treatment applied for migraines involve the application of responsive drugs and the identification of “triggers”—conditions unique to the human subject that trigger (e.g., initiate, cause, invoke, or activate) the migraine headache to begin. Such drugs commonly prescribed to migraine headache sufferers do not cure the migraine but only moderate (sometimes to a limited degree) the effects of the headache. Thus, long-term improvement relies heavily on the identification of triggers and changes to behavior and environment for the specific human subject.

However, identification of migraine triggers is very difficult. This is because, among other reasons, migraine triggers vary among different human subjects. Additionally, triggers may include a wide variety of sources, triggers may vary over time, and triggers may be composite (e.g., result from more than one trigger or in response to multiple external conditions). Thus, current techniques for detecting, identifying, and reducing the incidence of migraines and associated triggers are limited, and resulting migraine conditions can be very difficult to isolate, test, and verify.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings in which:

FIG. 1 illustrates a migraine trigger detection processing use case involving a migraine trigger detection device, a wearable sensor, a mobile computing device, and a cloud-based data processing service, according to an example;

FIG. 2 illustrates a scenario of a migraine trigger detection device capturing data in the presence of a sound source, a light source, and an odor source, according to an example;

FIG. 3 illustrates a diagram of communications occurring among a trigger detection device, a computing device, a data processing system, and a user display, according to an example;

FIG. 4 illustrates a flowchart of a method performed for capturing and analyzing potential migraine trigger data among multiple migraine episodes, according to an example;

FIG. 5 illustrates a flowchart of a method performed for capturing and analyzing data from a particular migraine episode, according to an example;

FIG. 6 illustrates a flowchart of a method performed by a computing device for processing and interacting with migraine trigger data via various computing device inputs and outputs, according to an example;

FIG. 7 illustrates a block diagram for an example system including a migraine trigger detection device, computing device, trigger processing component, and an optional external sensor device, according to an example;

FIG. 8 illustrates a block diagram for an example computer system architecture upon which any one or more of the techniques (e.g., operations, processes, methods, and methodologies) discussed herein may be performed, according to an example.

### DETAILED DESCRIPTION

In the following description, methods, configurations, device components, and related apparatuses are disclosed that provide for data processing used in the detection of migraine triggers or similar human pain conditions. Specifically, the following description includes various examples of a migraine trigger detection device that is configured to collect data on ambient stimuli, through a plurality of various sensors, and a computing device connected to the migraine trigger detection device that is configured to receive and process the data and correlate the detected stimuli with migraine symptoms. The following description also provides examples of processing activities that may occur with use of a cloud-based data processing system, including the collection, correlation, and analysis of data from multiple migraine episodes. Further, the following description also provides examples of data collection and input/output activities on the computing device, and data collection and analysis assisted with the use of related personal electronic devices (such as wearable devices that include physiological or environmental data sensors).

Many possible triggers for migraines are environmental in nature, such as noise, light conditions or patterns, and odors. Other types of possible triggers for migraines may include food or drink, medications, stress, and sleep patterns. As part of existing medical treatments, a human subject is often encouraged to keep a manual “log” of all of these conditions and attempt to identify commonalities that were present prior to the onset of a migraine. This is extremely difficult and error prone. The human subject often lacks the ability to identify some of the conditions sufficiently (such as sleep patterns, light patterns), or the human subject may lack the ability to track and identify composite patterns of triggers and correlate which triggers resulted in a migraine condition. The techniques discussed herein provide a data-oriented approach to obtain and process such conditions and provide a useable output to help prevent, mitigate, and identify migraine symptoms and the combination of triggers that cause such migraines.

As discussed herein, various types of computing devices and electronic devices equipped with sensors may be involved in data collection and analytics for the evaluation of ambient environment data from stimuli. The collection and analytics of ambient environment data that affects human sensory inputs (such as data indicating levels, patterns, or types of sound, light, and odors) may be used to generate reports, alerts, and analysis on the types of ambient stimuli that are relevant to migraine triggers. Further, the collection and analytics of such ambient environment data may be used to alert a human subject to the incidence of such migraine triggers (even if they are not being perceived by the human subject), to allow the human subject to take action to avoid or reduce exposure to such triggers.

In an example, a wearable device, such as a head-worn apparatus may include optical, audio, or chemical sensors, allowing the apparatus to sample and record ambient stimuli levels, events, and patterns in proximity to a human subject. Such stimuli levels may record light levels to record an approximation of what a human subject's eye is seeing, gas samples to record an approximation of what the human subject's nose is smelling, and noise levels and patterns to record an approximation of what the human subject's ear is hearing. Such stimuli data may be coupled with data from other wearables (e.g., a wrist-worn apparatus) that can track behavior data (e.g., sleep data) and physiological data (e.g., heart rate, perspiration), and data from user input (e.g. diet data entered via a smartphone). This stimuli data may be processed directly at a computing device with associated output and recommendations provided with a user interface of the computing device.

Thus, with use of the present techniques, many possible triggers for migraine headaches or like pain conditions may be tracked, in addition to the tracking of relevant time data, physiological data, and user data. In further examples, the stimuli data may be collected on a smartphone or mobile computing device, and communicated to a cloud-based data processing service that can perform a correlation analysis to help the human subject identify and test for possible triggers. And in still further examples, the cloud-based data processing service may use data collected across aggregates of many users, which may identify triggers that are population based (based on, or independent of, gender, race, region, environment, medication, etc.).

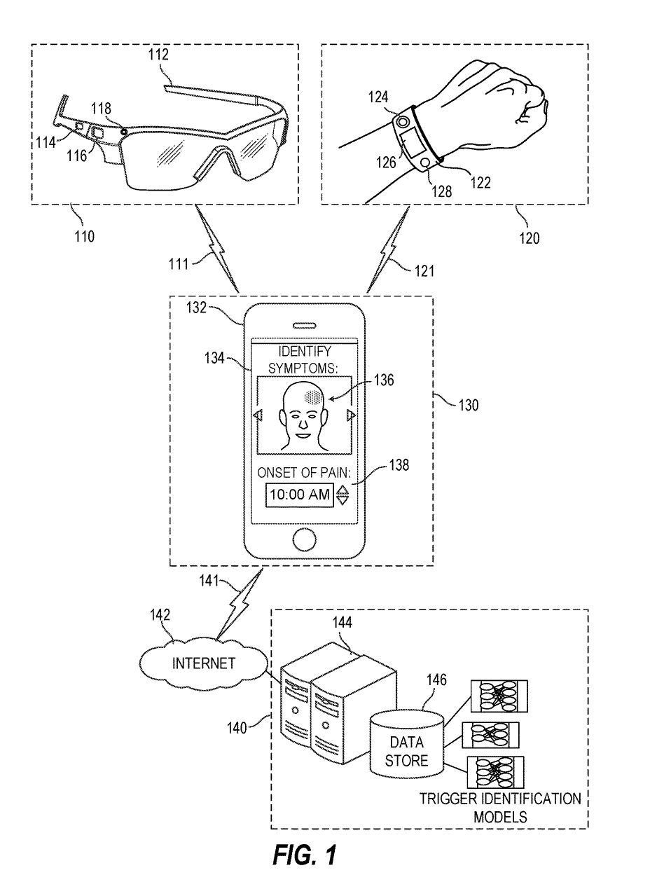

FIG. 1 illustrates a migraine trigger detection processing use case for use with a human subject, according to an example device configuration. As shown, the migraine trigger detection processing use case involves a migraine trigger detection device 110 (e.g., glasses), a physiological sensor device 120 (e.g., a wrist wearable device), a computing device 130 (e.g., a smartphone), and a cloud-based remote processing system 140. These electronic devices and subsystems operate to assist the identification of migraine triggers as described herein. However, it will be understood that more or fewer devices may be employed for analysis, identification, data processing, or evaluation of migraine triggers or like pain conditions.

As shown, the migraine trigger detection device 110, the physiologic sensor device 120, the computing device 130, and the cloud-based remote processing system 140 are physically separate electronic devices that are configured to communicate with each other (e.g., via wireless communications). In some examples, the features of the migraine trigger detection device 110 and the computing device 130 may be integrated into a single device (e.g. a migraine trigger detection device with increased computing power and the ability to project a display directly to a user). In other examples, the functions of the respective devices may be further segmented and divided among separate components or modules.

The migraine trigger detection device 110 of FIG. 1 is shown in the form factor of a wearable glasses headset 112, including a first sensor 114 for sound detection, a second sensor 116 for odor compound detection, and a third sensor 118 for light detection. The migraine trigger detection device 110, however, may take a variety of forms, and include additional or fewer sensors, or may be integrated into a different type of wearable or personal device form factor such as a watch, handheld device, into clothing or accessories, or the like.

The physiologic sensor device 120 of FIG. 1 is shown in the form factor of a wrist worn wearable sensor unit 122, but the physiologic sensor device 120 may take on other form factors (e.g. a chest strap heart rate monitor, a food sensor placed internally within a human user, etc.). The wearable sensor unit 122 may include one or more physiologic data sensors, such as a human characteristic sensor 124 (e.g., heart rate, perspiration monitor), an environment sensor (not shown, e.g., accelerometer, gyroscope, a strain gauge, a proximity sensor, or a temperature sensor), in addition to an output screen 126, and an output component 128 (e.g., an LED).

The physiologic sensor device 120 may be used to provide physiologic data relevant a current or historical state of the human subject. For example, such physiologic data may include sleep data (like a Basis Peak), heart rate, blood oxygen rate, and perspiration rate (e.g., through measurements of galvanic skin response (GSR) or other electrodermal activity).

The computing device 130 of FIG. 1 is shown in the form factor of a smartphone 132 including a display 134. However, in other examples the computing device 130 could be a smartwatch, eyeglass screen, or the like. In another example, the computing device 130 could be a laptop, tablet, personal computer, thin client, or like computing device. The computing device 130 is shown as communicating with the migraine trigger detection device 110 via wireless connection 111 and with physiologic sensor device 120 via wireless connection 121, such as to receive stimuli and physiological data respectively.

The computing device 130 is further shown as including a user interface output on the display 134. The user interface may be used to receive user feedback regarding a positive weighting of an identified migraine trigger (e.g., to indicate that the trigger causes specific symptoms) or to receive user feedback regarding a negative weighting of an identified migraine trigger (e.g., to indicate that the trigger occurs under non-migraine conditions). In an example, the user interface may include a graphical input interface 136 to receive an indication of symptoms, and a time input interface 138 to receive an indication of time or other characteristics of symptoms. The user interface may be provided through standalone software, a platform-specific application (app), a cloud or web-hosted application, or the like. Additionally, the user input via a smartphone may be used to indicate possible triggers, symptoms, intensity of such triggers and symptoms, as perceived by the human subject. Further, such user input may include information such as diet data, or response to other episodic questions or suggestions (with time data as relevant).

The cloud-based remote processing system 140 of FIG. 1 is shown as connected to the computing device 130 via a connection 141 (e.g., a wide area wired or wireless connection) over the Internet 142 (or other suitable network). The cloud-based remote processing system 140 may include one or a plurality of computing devices 144 and one or a plurality of data stores 146 (and accompanying databases) to persist, stage, and host data. The cloud-based remote processing system 140 may store, host, or access a number of trigger identification models that are used to process stimuli data and identified characteristics of a migraine symptom occurrence. Thus, the cloud-based remote processing system 140 may be used to assist the migraine symptom and trigger identification process that is output with the computing device 130.

In an example, the data processing of respective stimuli correlations and trigger identifications performed in the environment of FIG. 1 is performed at the cloud-based remote processing system 140, with the use of various communications exchanged via the Internet 142. In a further example, such correlations and identifications may be offloaded to the computing device 130, or integrated in part within the migraine trigger detection device 110 (based on the hardware processing capabilities of the detection device). Additionally, in some examples, the migraine trigger detection device 110, the physiologic sensor device 120, or other wearables might communicate directly with the cloud-based remote processing system 140. Communications may also be performed among respective devices in to assist data processing for multiple users in an area, such as coordination of common migraine triggers among multiple human subjects.

In response to the data received, and the processing of stimuli data (such as processing with the cloud-based remote processing system 140, the computing device 130, or the migraine trigger detection device 110), the graphical user interface (provided on the display 134) may identify and output triggers that have a high correlation to migraine events. A variety of suggestions, instructions, recommendations, warnings, and indications may be output with the graphical user interface on the display 134. For example, user outputs may provide information to a human subject about active triggers, therapy or pain management, and typical migraine symptoms and phases. Additionally, such identified triggers may result in other electronic actions, such as automated activation of a medical or therapy device, as discussed herein.

In an example, the cloud-based remote processing system 140 may perform pattern matching algorithms to a time-varied set of data, to identify suggested and probable correlations between stimuli and migraine events for a particular human subject. This type of pattern matching and data recording is impractical (and impossible) to fully conduct by the human subject, as a human subject is not able to observe all stimuli conditions. As a result, the present migraine trigger analysis techniques are data-driven and based on a rigorous evaluation of quantified data. Further, the present migraine trigger analysis techniques are sensor-driven, rather than being based on human memory. The use of this data analysis is quantified to a sufficient level to provide a high level of precision and granularity of migraine conditions and stimuli triggers, while considering stimuli triggers that would otherwise be unperceivable by a human subject.

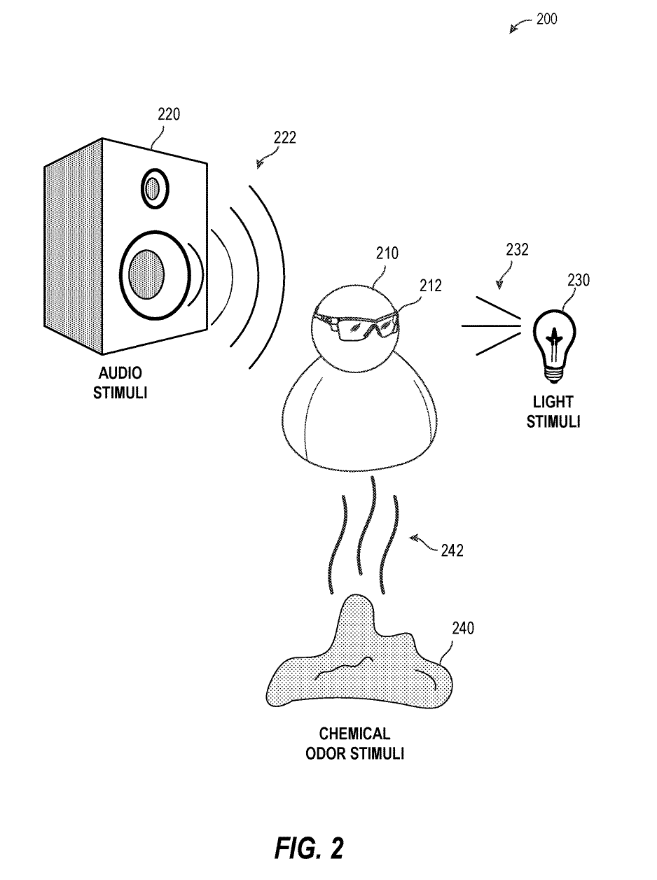

FIG. 2 illustrates an example scenario 200 of a migraine trigger detection device 212 capturing data in the presence of a sound source 220, a light source 230, and an odor source 240. As illustrated, the example scenario 200 includes a human subject 210 wearing a migraine trigger detection device 212, which is integrated into a wearable object such as the depicted glasses. The human subject 210 is located in an environment with the sound source 220 emitting varying levels of audio stimuli 222, the light source 230 emitting varying levels of light stimuli 232, and the odor source 240 emitting varying levels of odor stimuli 242. (Although only one source is depicted, additional or complex sources may provide intensity and changes to the respective levels of stimuli).

The migraine trigger detection device 212 operates to collect stimuli characteristics, regarding the timing, quantity, and strength (and patterns and variations) of the various stimuli. Specifically, the ambient sensors included on the migraine trigger detection device 212 collect data relevant to the stimuli characteristics, and communicate this data to a computing device (not shown) of the human subject 210. In response to the audio stimuli 222, the light stimuli 232, or the odor stimuli 242, the migraine trigger detection device 212 operates to generate stimuli data and communicate such stimuli data to a connected computing device. The connected computing device then may be used to analyze the stimuli data, communicate the stimuli data to a remote processing service, and the like.

In an example, the stimuli data is periodically transmitted from the migraine trigger detection device 212 to a connected computing device (such as a smartphone, when within range). When a migraine occurs, the user indicates a pain condition using an application (e.g., smartphone app), with the user inputting an estimated time that the symptoms began, and a current stage of the migraine (e.g., Prodromal, Aural, Attack, Postdromal stages of a migraine). In a further example, the migraine trigger detection device 212 may perform sampling of its sensors based on per-sensor algorithms that are controlled by the analysis needs of a particular stimuli, the pain condition, and other input from a user (thus, controlling how often a camera is sampled to obtain light patterns, how and what data is sampled from a microphone to obtain audio intensity, etc.).

Data collected by the computing device that has passed some age window, without a migraine event being identified by the user, may be purged from the migraine trigger detection device 212 or the connected computing device. For example, data collected from days or weeks ago, where a migraine did not occur is not considered a likely trigger. When a migraine is identified, however, data is evaluated for a period prior to the event and categorized based on per-sensor algorithms (e.g., was there an “odor” detected, was there a “flashing light pattern” detected, was there “loud noise” detected, and the like). These attributes may be then added to an event record. This event record may be processed (e.g., by the connected computing device, or a cloud-based data processing service). Analysis may be performed using the historically collected set of event records, including event potentially weighted based on other groups, global biases, or other useful data values.

Triggers that are found to have a high correlation to events are then presented to the user. The user can then provide feedback regarding negative weighting (e.g. if that same trigger occurs under nonevent conditions). Additionally, the identified trigger or triggers can then be fed back to the device or smartphone algorithm for negative case monitoring. In future samples, if that trigger is identified but the user has not indicated a migraine event, its weight in the correlation analysis may be decreased. The user may be presented with listings and ratings of triggers to help assign stronger or lesser weights to particular stimuli.

Over time, the identification of high positive correlation (and low negative correlation triggers) can be identified to a user for further testing and verification. If (and when) a user is able to identify his or her triggers, the user may also be able to submit relevant data (including demographics, verification, and analysis) into a larger aggregate pool to help refine algorithms and improve general medical understanding of migraines. Such data aggregation may be subject to privacy controls, user consent and permission, to enable querying with the cloud-based data processing system.

Current techniques for tracking stimuli related to migraine headaches are manual and human-initiated processes, and are limited in effectiveness, because humans lack the ability to quantify most triggers. Further, many humans are not even able to identify triggers from stimuli, such as the type or strength of a particular odor. As a result, current techniques for identifying triggers in audio, light, or chemical stimuli are often error prone. Further, such existing techniques are unable to fully track the time of occurrence for triggers, and are unlikely to identify common possible triggers especially under composite conditions. The use of the presently described techniques enable an automated identification of correlation and causation for such triggers, allowing improvements to the functioning of relevant recommendation (and data processing) systems and algorithms, wearable devices, and logic deployed in medical and physiological devices.

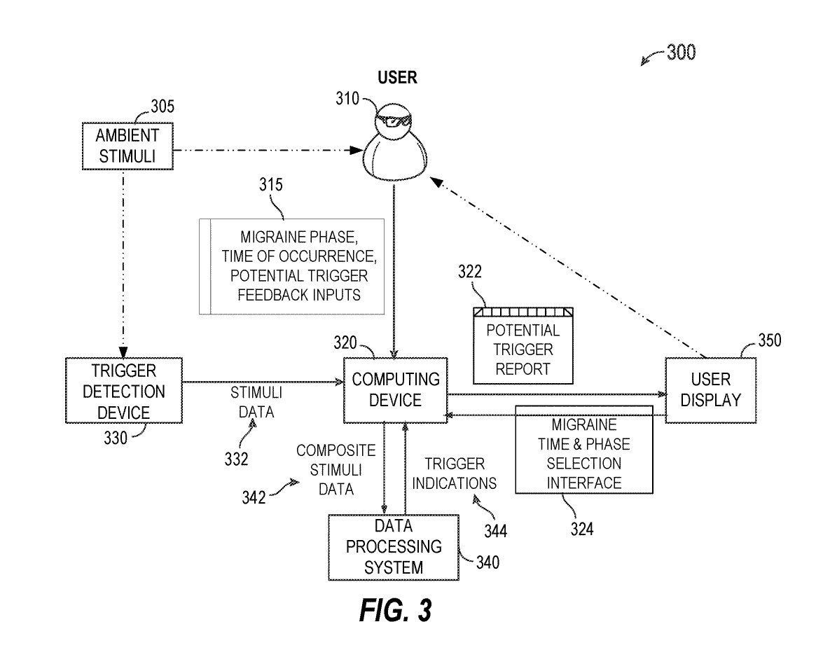

FIG. 3 illustrates a diagram of example communications occurring among a trigger detection device 330 worn or controlled by a human subject 310 (a user), a computing device 320, a data processing system 340, and a user display 350. As illustrated, the diagram of FIG. 3 includes example data flows 300, wherein the trigger detection device 330 senses ambient stimuli 305 in proximity to the human subject 310. This ambient stimuli 305 may include the stimuli described with reference to FIG. 2 above, or may include other forms of sensory stimuli that can be captured by a sensor and attributed to a data value.

The trigger detection device 330 may include one or more sensors such as a light sensing component, an odorous compound sensing component, or a sound sensing component, used to detect the ambient stimuli 305 occurring in the environment of the human subject 310. These sensors may be provided in the form of visible and infrared light sensors, gas detection sensors, sound sensors, embodied by microelectromechanical (MEMS) sensors, biosensors, or like components. Such sensors may record the data continuously, or in intervals, and communicate this stimuli data in real time or at respective intervals. The trigger detection device 330 may further include a camera or microphone to record audiovisual data related to the ambient stimuli 305 and environmental conditions.

The trigger detection device 330 produces a form of stimuli data 332 from observations of the ambient stimuli 305, and this stimuli data 332 is communicated to the computing device 320. The computing device 320 also receives input from the human subject 310 that relates to the incidence of a migraine phase and the time of occurrence, as well as potential trigger feedback. This data may be communicated wirelessly using any number of personal or local area protocols (e.g., Bluetooth, Wi-Fi), or via a wide area connection (e.g., via a mobile network/4G data connection).

The computing device 320 may provide output and receive input with a user display 350 (e.g. a touchscreen of a smartphone). The user display 350 may provide a user interface (e.g., graphical user interface) for outputting a potential trigger report 322, and receive a user input 315 of a migraine phase and a time of occurrence (such as in a time and phase selection interface 324).

The computing device 320 may provide composite stimuli data 342 to a data processing system 340, such as composite stimuli data 342 filtered and processed by the computing device 320. The data processing system 340 may then return identified trigger indications 344 or other relevant content, suggestions, and data points. In some examples, the computing device 320 may provide raw or filtered raw data to the data processing system that is obtained from the migraine trigger detection device 310.

The data flows 300 that occur in the depicted system may be based on the detected phase of the migraine that is indicated by the human subject 310 or detected by the computing device 320. For example, a migraine that is in an early phase may be alerted to the human subject 310, along with a series of questions in the time and phase selection interface 324 to inquire about the state of the migraine. However, a migraine that is in a later phase may be indicated by the human subject 310, and the data processing system 340 may attempt to identify the specific trigger that for a surrounding time period, the phases in which the triggers were active, and like information.

The information collected with the data flows 300 also may be used to determine prior triggers. For prior migraine episodes, the respective input choices in the interface 324 and other user interactions may be used to locate relevant data points relevant to a migraine episode. For example, if a user remembers and indicates that a trigger occurred at 1 PM, the computing device 320 and the data processing system 340 can perform analysis to determine what the most likely trigger was. Spanning an hour back, or another time window, a user can access and identify multiple data points, combinations of triggers, phases of the migraine, and the like. Such data correlation and processing is not possible with human or manual techniques.

The data processing system 340 may apply a machine analysis for detecting triggers using multivariable conditions. A number of machine learning (e.g., deep learning), training, and recognition algorithms may be performed to identify and improve trigger recognition. In addition, the data processing system 340 may apply models that are trained to be specific to the human subject, or that are specific to a population of subjects (or a universe of subjects). For example, the data processing system 340 may utilize a global model, to identify certain patterns that could be optimized from many contributions, and to identify use cases that are not previously recognized (such as a new chemical signature that was not previously recognized as a trigger). Also for example, the data model may be trained towards specific data and data types based on population characteristics and commonalities.

In further examples, as a result of the trigger identification, various electronic actions may occur in the trigger detection device 330, an associated wearable device, the computing device 320, or other controllable or interactive systems. In addition to the user interface use cases discussed above, such electronic actions may include counteraction measures to help reduce the symptoms of a migraine, such as: activation of lenses of glasses that can darken and adapt to filter or reduce light; activation of ear filters or ear buds (or counteracting audio sounds) to filter or reduce sound; activation of smell emission devices to counteract smells. The counteraction measures may also invoke forms of drug or therapy devices, such as to control the amount of drug or physiologic therapy provided from a controllable medical device.

Also in further examples, other forms of physiologic sensors may be used for providing data inputs to the data flows 300 of FIG. 3. Such physiologic sensors may include the wearable sensors previously described, in addition to other medical monitoring sensors such as pain sensors, brain activity (e.g., EEG) sensors, and the like. Further, medical diagnostic sensors may be used to detect neural patterns, pain conditions, and formation of a migraine in parts of the head anatomy, to initiate identification of pain triggers with the trigger detection device. Likewise, other forms of internal or external sensors may be used, such as a food digestion sensor to detect exposure to certain chemicals, or a sensor in a tooth to detect characteristics of food. Other forms of biologic, chemical, or physiological sensor data may be used to assist identification of the trigger for the migraine and what phase that the migraine is occurring (or the severity of symptoms).

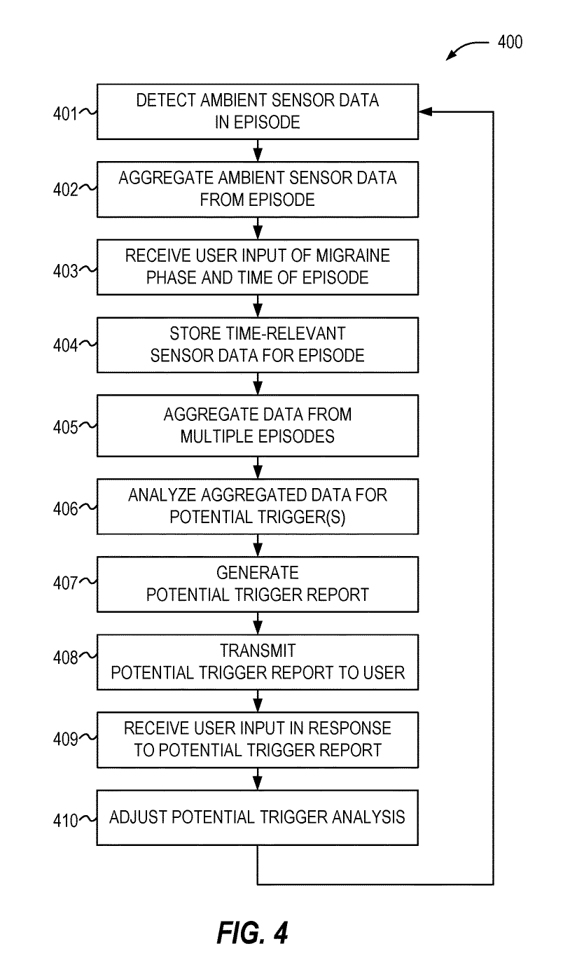

FIG. 4 illustrates a flowchart 400 of an example method performed for capturing and analyzing potential migraine trigger data among multiple episodes. The flowchart 400 provides a general overview of the collection and analysis of data from an ambient sensor data device, such as one or more ambient sensors included in a migraine trigger detection device. Thus, it will be apparent that the data processing techniques and data flows described above with reference to FIGS. 1 to 3 may also be incorporated into the method of the flowchart 400.

As depicted, the flowchart 400 includes detecting ambient sensor data (operation 401) such as light, sound, odors, and like ambient stimuli, as part of detection of a migraine episode. Such migraine episode may include a period of time indicated by a user, detected by stimuli data values, or identified on an ongoing basis. During the episode, ambient sensor data from the episode (e.g., for a predetermined time period) is aggregated and collected (operation 402) from a migraine trigger detection device.

The data may be consistently recorded by a migraine trigger detection device, and identified as part of a migraine episode in response to receiving user input of a migraine episode (such as migraine phase or severity) and time of occurrence (operation 403). In connection with the identification or detection of a migraine episode, relevant time-sensor sensor data is processed and stored with a computing device connected to the migraine trigger detection device (operation 404). This is accompanied by the aggregation of data from multiple migraine episodes (operation 405), and in some examples, the communication of this aggregated data to a cloud-based data processing system.

The aggregated data from multiple migraine episodes is analyzed for potential migraine triggers (operation 406), such as with the analysis of recurring sensor data inputs proximate to the time or phase of a particular migraine episode occurrence. Based on this analysis, a report may be generated that identifies the potential triggers (operation 407), and this potential trigger report may be transmitted to a user's computing device, or otherwise output to the user via an output medium (operation 408). For example, the potential trigger report may be communicated from the cloud-based data processing system to a computing device, to be output via a graphical user interface.

In response to the potential trigger report, the computing device may receive user input containing feedback on the potential migraine triggers reported to the user (operation 409). This feedback may be used to confirm or deny particular triggers and migraine symptoms, which is used to adjust the potential trigger analysis based upon the user input received (operation 410). The process may recursively continue, identifying likely migraine triggers and migraine pain events using multiple episodic data that is captured with a migraine trigger detection device on an ongoing basis. In some examples, the multiple episodic data that is processed and analyzed may originate from other types of historical data for the subject, another subject, or groups of subjects.

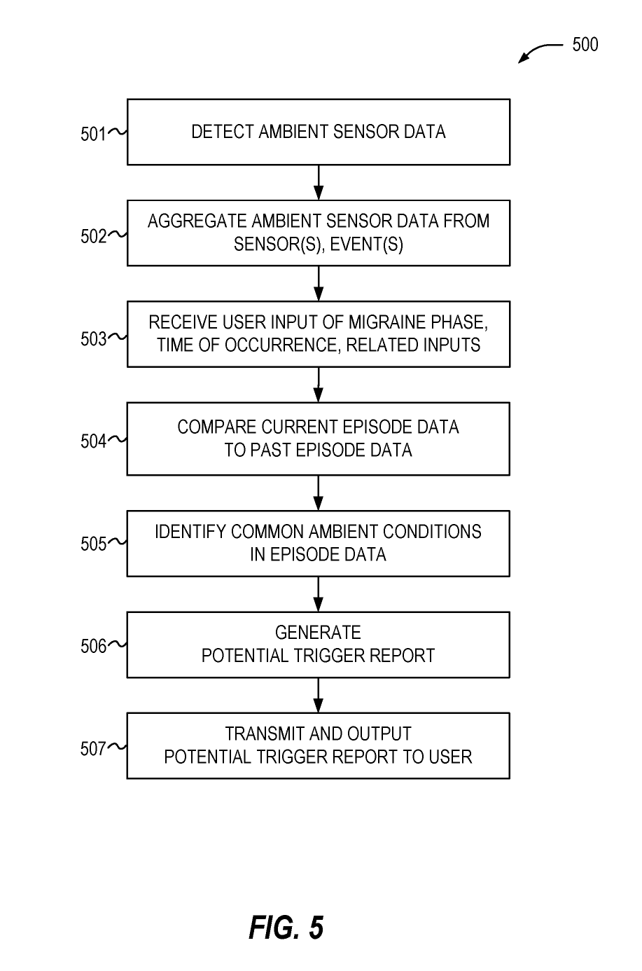

FIG. 5 illustrates a flowchart 500 of an example method performed for capturing and analyzing data from a particular migraine episode. Specifically, the flowchart 500 depicts a use case for the capture of data with use of an ambient stimuli sensor integrated into a migraine trigger detection device. Thus, the method depicted in the flowchart 500 may be performed by operations of a migraine trigger detection device, a computing device in communication with the detection device, a remote processing system in communication with the detection device or computing device, or some combination of these devices or systems. Moreover, the data processing techniques and data flows described above with reference to FIGS. 1 to 3 may also be incorporated into the method of the flowchart 500.

The migraine trigger detection device operates to detect ambient stimuli sensor data (operation 501) from one or more sensors on an ongoing basis, and such sensor data from multiple events or time intervals is aggregated (operation 502). The migraine trigger detection device or a connected computing device receives user input indicating a migraine episode with the time of occurrence, and the identification of relevant migraine characteristics (such as migraine phase) (operation 503). Sensor data that is relevant to the occurrence of the current migraine episode (current episode data) is further processed, filtered, communicated, and stored, so that it can be compared to sensor data from one or more past migraine episodes (past episode data). In some examples, this data processing is performed with use of a cloud-based data processing system that retains the historical data and operates the correlation analysis process.

Based on the receipt of the current episode data, the current episode data is compared to past episode data (operation 504). Similar ambient conditions, trigger characteristics, and migraine or stimuli properties are identified from the comparison (operation 505). The identification of the similar ambient conditions are then used to generate a potential trigger report (operation 506). This trigger report may be transmitted to a user's computing device, or otherwise output to the user via an output medium (operation 507). For example, the potential trigger report may be communicated from the cloud-based data processing system to a computing device, to be output via a graphical user interface.

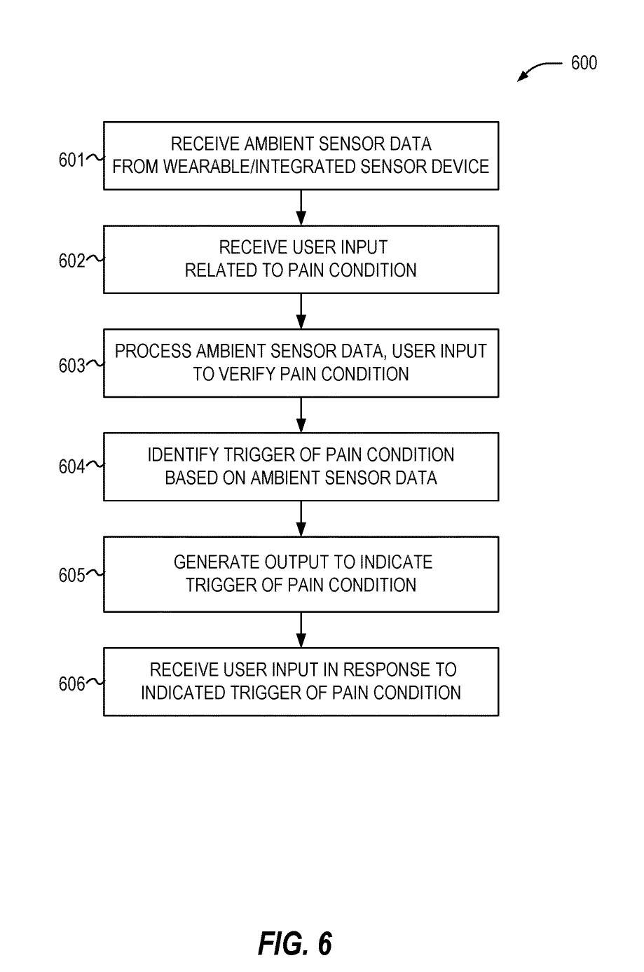

FIG. 6 illustrates a flowchart 600 of an example method performed by a computing device for processing and interacting with migraine trigger data via various computing device inputs and outputs. The flowchart 600 is illustrated from the perspective of the computing device (such as a connected smartphone) which may operate specialized software applications to implement the features of the method. However, features of the method may be performed in part by a remote processing system or of the connected detection device. Thus, variations to the flowchart 600 may include the data processing techniques, data flows, or operations described above with reference to FIGS. 1 to 5.

As illustrated, the flowchart 600 includes a sequence of events involving the communication of data relevant to a potential migraine event, to a connected computing device (e.g., a personal computer, tablet, smartphone, etc.). This sequence events starts with the receipt or capture of ambient sensor data from a wearable or integrated sensor device (operation 601) and the receipt or capture of user input related to the migraine event (operation 602). This user input may be received using a graphical user interface as previously described. The connected computing device may process this ambient sensor data and user input to verify the migraine pain condition (operation 603) directly, or involve the use of a remote processing service or external data source.

Based on the characteristics indicated by a received user input and ambient sensor data, the connected computing device operates to identify a particular trigger of a pain condition (operation 604). This identified trigger is then included in output from the connected computing device (operation 605), such as a graphical output which indicates a particular type of stimuli that is most strongly correlated to the trigger. The connected computing device may receive further user input in response to this indicated trigger of the migraine, such as confirmation that the trigger is or is not relevant, an updated status of pain, recommendations related to the trigger type, and the like.

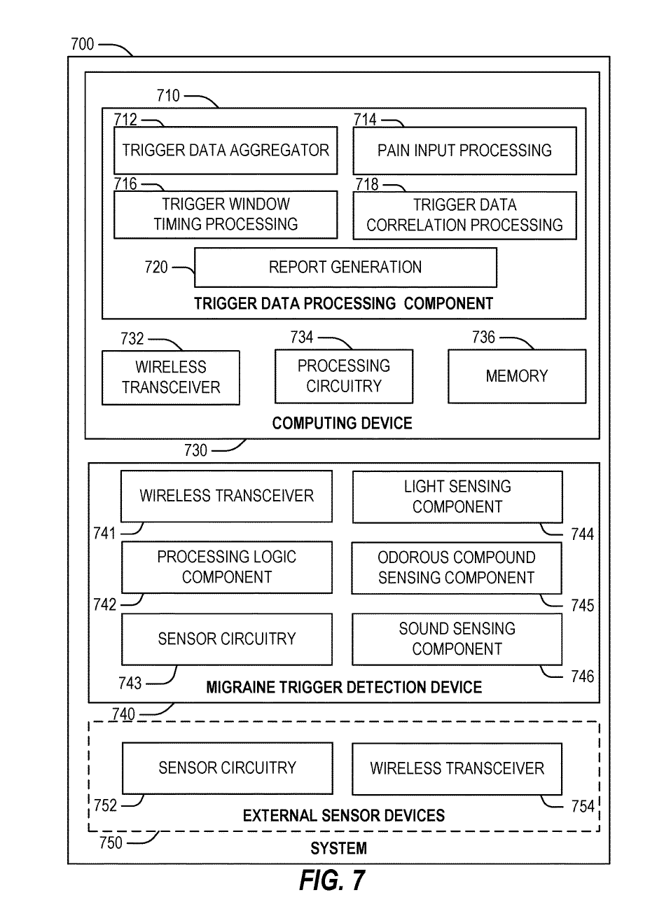

FIG. 7 is a block diagram illustrating an example system 700 including a migraine trigger detection device 740, a computing device 730 further containing a trigger data processing component 710, and an optional external sensor device 750. The components and devices of the system 700 may be used to implement the features described above with reference to FIGS. 1 to 7.

The migraine trigger detection device 740 is depicted as including a wireless transceiver 741, a processing logic component 742, sensor circuitry 743, a light sensing component 744, an odorous compound sensing component 745, and a sound sensing component 746. For example, the processing logic component 742 may operate to directly identify and indicate a trigger of the migraine headache condition, based on a level, a pattern, and a type of a particular stimulus of the plurality of ambient stimuli. In other examples, the processing logic component 742 may operate to compile data from multiple of the ambient stimuli sensors and communicate the data to the computing device 730.

The computing device 730 is depicted as including a wireless transceiver 732, processing circuitry 734, memory 736, and a trigger data processing component 710. The trigger data processing component 710 is depicted as including a trigger data aggregator 712, a pain input processing component 714, a trigger window timing processing component 716, a trigger data correlation processing component 718, and a report generation component 720. For example, the trigger data processing component 710 may operate to directly process sensor data for trigger identification, or process and communicate features of the sensor data to a remote processing system for trigger identification. For instance, the trigger data aggregator 712 may identify respective conditions of a plurality of ambient stimuli, from the sensor data, that occur in proximity to the human subject; the pain input processing component 714 may evaluate input received from the human subject that indicates a migraine headache condition; the trigger window timing processing component 716 may identify a time interval relevant to the migraine headache condition of the human subject; the report generation component 720 may generate a trigger report to output to the human subject; and he trigger data correlation processing component 718 may process the sensor data and the received input to identify the stimuli trigger of the migraine headache condition.

The external sensor device 750 (which may include one or more device) is depicted as including sensor circuitry 752, and a wireless transceiver 754. The sensor circuitry 752 may operate to collect and process the physiological data from at least one sensor, and communicate this data to the computing device 730 using the wireless transceiver 754. In some examples, the system 700 does not include use of the external sensor device 750.

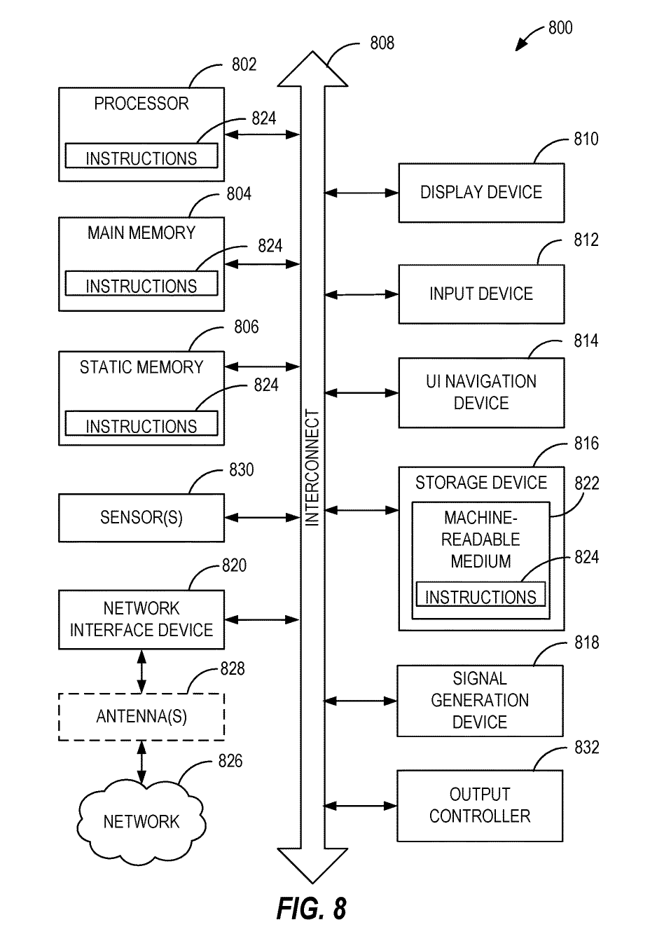

FIG. 8 is a block diagram illustrating a machine in the example form of a computing system (e.g., computing device) 800, within which a set or sequence of instructions may be executed to cause the machine to perform, implement, or execute operations for any one of the methodologies discussed herein, according to an example embodiment. The machine may be a personal computer (PC), a tablet PC, a hybrid tablet/notebook PC, a personal digital assistant (PDA), a mobile telephone or smartphone, a wearable computer or device, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 800 includes at least one processor 802 (e.g., a central processing unit (CPU), a graphics processing unit (GPU) or both, processor cores, compute nodes, etc.), a main memory 804 and a static memory 806, which communicate with each other via an interconnect 808 (e.g., a link, a bus, etc.). The computer system 800 may further include a video display unit 810, an alphanumeric input device 812 (e.g., a keyboard), and a user interface (UI) navigation device 814 (e.g., a mouse). In one embodiment, the video display unit 810, input device 812 and UI navigation device 814 are incorporated into a touch screen display. The computer system 800 may additionally include a storage device 816 (e.g., a drive unit), a signal generation device 818 (e.g., a speaker), an output controller 832, a network interface device 820 (which may include or operably communicate with one or more antennas 828, transceivers, or other wireless communications hardware), and one or more sensors 830, such as a global positioning system (GPS) sensor, compass, accelerometer, location sensor, or other sensor.

The storage device 816 includes a machine-readable medium 822 on which is stored one or more sets of data structures and instructions 824 (e.g., software) embodying or utilized by any one or more of the methodologies or functions described herein. The instructions 824 may also reside, completely or at least partially, within the main memory 804, static memory 806, and/or within the processor 802 during execution thereof by the computer system 800, with the main memory 804, static memory 806, and the processor 802 also constituting machine-readable media.

While the machine-readable medium 822 is illustrated in an example embodiment to be a single medium, the term “machine-readable medium” may include a single medium or multiple media (e.g., a centralized or distributed database, and/or associated caches and servers) that store the one or more instructions 824. The term “machine-readable medium” shall also be taken to include any tangible medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 824 may further be transmitted or received over a communications network 826 via an antenna 828 using a transmission medium via the network interface device 820 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 2G/3G, and 4G LTE/LTE-A or WiMAX networks). The term “transmission medium” shall be taken to include any intangible medium that is capable of storing, encoding, or carrying instructions for execution by the machine, and includes digital or analog communications signals or other intangible medium to facilitate communication of such software.

Embodiments used to facilitate and perform the techniques described herein may be implemented in one or a combination of hardware, firmware, and software. Embodiments may also be implemented as instructions stored on a machine-readable storage device, which may be read and executed by at least one processor to perform the operations described herein. A machine-readable storage device may include any non-transitory mechanism for storing information in a form readable by a machine (e.g., a computer). For example, a machine-readable storage device may include read-only memory (ROM), random-access memory (RAM), magnetic disk storage media, optical storage media, flash-memory devices, and other storage devices and media.

It should be understood that the functional units or capabilities described in this specification may have been referred to or labeled as components or modules, in order to more particularly emphasize their implementation independence. Such components may be embodied by any number of software or hardware forms. For example, a component or module may be implemented as a hardware circuit comprising custom very-large-scale integration (VLSI) circuits or gate arrays, off-the-shelf semiconductors such as logic chips, transistors, or other discrete components. A component or module may also be implemented in programmable hardware devices such as field programmable gate arrays, programmable array logic, programmable logic devices, or the like. Components or modules may also be implemented in software for execution by various types of processors. An identified component or module of executable code may, for instance, comprise one or more physical or logical blocks of computer instructions, which may, for instance, be organized as an object, procedure, or function. Nevertheless, the executables of an identified component or module need not be physically located together, but may comprise disparate instructions stored in different locations which, when joined logically together, comprise the component or module and achieve the stated purpose for the component or module.

Indeed, a component or module of executable code may be a single instruction, or many instructions, and may even be distributed over several different code segments, among different programs, and across several memory devices. Similarly, operational data may be identified and illustrated herein within components or modules, and may be embodied in any suitable form and organized within any suitable type of data structure. The operational data may be collected as a single data set, or may be distributed over different locations including over different storage devices, and may exist, at least partially, merely as electronic signals on a system or network. The components or modules may be passive or active, including agents operable to perform desired functions.

Additional examples of the presently described method, system, and device embodiments include the following, non-limiting configurations. Each of the following non-limiting examples may stand on its own, or may be combined in any permutation or combination with any one or more of the other examples provided below or throughout the present disclosure.

Example 1 is a computing device, comprising processing circuitry to: receive sensor data from a sensor device worn by a human subject, wherein the sensor data indicates respective conditions of a plurality of ambient stimuli occurring in proximity to the human subject; receive input from the human subject, wherein the input indicates a pain condition of the human subject; process the sensor data and the received input with a user-trained model to identify a trigger of the pain condition, wherein the trigger of the pain condition corresponds to a particular stimulus of the plurality of ambient stimuli; and generate output to the human subject, wherein the output indicates the trigger of the pain condition.

In Example 2, the subject matter of Example 1 optionally includes wherein the pain condition of the human subject is a migraine headache, wherein the trigger of the pain condition relates to an environmental characteristic of the particular stimulus, and wherein the generated output includes an identification of the environmental characteristic of the particular stimulus as the trigger of the migraine headache.

In Example 3, the subject matter of Example 2 optionally includes wherein the operations that process the sensor data and the received input are used to identify the trigger of the migraine headache from a plurality of potential trigger conditions indicated in the sensor data, wherein the plurality of potential triggers are identified from sensor data produced for the plurality of ambient stimuli, and wherein the trigger is identified based on a degree of correlation of the trigger in a past occurrence of a migraine headache with the human subject.

In Example 4, the subject matter of any one or more of Examples 2-3 optionally include wherein the received input further includes feedback from the user of a negative weighting of other stimuli characteristics from the plurality of ambient stimuli, and wherein, based on the negative weighting, the other stimuli characteristics from the plurality of ambient stimuli are identified as not being the trigger of the migraine headache.

In Example 5, the subject matter of any one or more of Examples 3-4 optionally include the processing circuitry further to: receive additional input from the human subject, wherein the input and the input is received in a graphical user interface that is provided on a display of the computing device, wherein the input indicates a phase of the migraine headache, and wherein the additional input indicates a time of occurrence of a particular phase of the migraine headache; and determine a time period corresponding to the particular phase of the migraine headache; wherein the trigger of the migraine headache is identified based on sensor data captured in the time period corresponding to the particular phase of the migraine headache.

In Example 6, the subject matter of Example 5 optionally includes a wireless transceiver to wirelessly communicate with a physiological sensor device; wherein the physiological sensor device includes a physiological sensor to produce physiologic sensor data captured from physiologic characteristics of the human subject; and wherein the trigger of the migraine headache is further identified based on the physiologic sensor data.

In Example 7, the subject matter of Example 6 optionally includes wherein the physiologic sensor data indicates one or more of: heart rate, perspiration, sleep patterns, or diet, of the human subject.

In Example 8, the subject matter of any one or more of Examples 3-7 optionally include a wireless transceiver to wirelessly communicate with the sensor device using a wireless communication protocol; wherein the operations to receive the sensor data from the sensor device are performed using the wireless communication protocol.

In Example 9, the subject matter of any one or more of Examples 1-8 optionally include the processing circuitry further to: communicate the sensor data to a remote data processing system; and receive an indication of the trigger of the pain condition from the remote data processing system, in response to communication of the sensor data and analysis by the remote data processing system; wherein the remote data processing system applies a trigger identification model to identify the trigger from the sensor data.

In Example 10, the subject matter of any one or more of Examples 1-9 optionally include wherein the plurality of ambient stimuli relates to at least two of light, sound, or odor, and wherein the sensor data indicates a level, a pattern, and a type for respective stimulus of the plurality of ambient stimuli.

Example 11 is a method, comprising by electronic operations executed in processing circuitry of a computing device, wherein the electronic operations include: receiving sensor data from a sensor device worn by a human subject, wherein the sensor data indicates respective conditions of a plurality of ambient stimuli occurring in proximity to the human subject; receiving input from the human subject, wherein the input indicates a pain condition of the human subject; processing the sensor data and the received input with a user-trained model to identify a trigger of the pain condition, wherein the trigger of the pain condition corresponds to a particular stimulus of the plurality of ambient stimuli; and generating output to the human subject, wherein the output indicates the trigger of the pain condition.

In Example 12, the subject matter of Example 11 optionally includes wherein the pain condition of the human subject is a migraine headache, wherein the trigger of the pain condition relates to an environmental characteristic of the particular stimulus, and wherein the generated output includes an identification of the environmental characteristic of the particular stimulus as the trigger of the migraine headache.

In Example 13, the subject matter of Example 12 optionally includes wherein the electronic operations that process the sensor data and the received input are used to identify the trigger of the migraine headache from a plurality of potential trigger conditions indicated in the sensor data, wherein the plurality of potential triggers are identified from sensor data produced for the plurality of ambient stimuli, and wherein the trigger is identified based on a degree of correlation of the trigger in a past occurrence of a migraine headache with the human subject.

In Example 14, the subject matter of any one or more of Examples 12-13 optionally include wherein the received input further includes feedback of a negative weighting of other stimuli characteristics from the plurality of ambient stimuli, and wherein, based on the negative weighting, the other stimuli characteristics from the plurality of ambient stimuli are identified as not being the trigger of the migraine headache.

In Example 15, the subject matter of any one or more of Examples 13-14 optionally include the electronic operations further including: receiving additional input from the human subject, wherein the input and the input is received in a graphical user interface that is provided on a display of the computing device, wherein the input indicates a phase of the migraine headache, and wherein the additional input indicates a time of occurrence of a particular phase of the migraine headache; and determining a time period corresponding to the particular phase of the migraine headache; wherein the trigger of the migraine headache is identified based on sensor data captured in the time period corresponding to the particular phase of the migraine headache.

In Example 16, the subject matter of Example 15 optionally includes the electronic operations further including: wirelessly communicating with a physiological sensor device; wherein the physiological sensor device includes a physiological sensor to produce physiologic sensor data captured from physiologic characteristics of the human subject; and wherein the trigger of the migraine headache is further identified based on the physiologic sensor data.

In Example 17, the subject matter of Example 16 optionally includes wherein the physiologic sensor data indicates one or more of: heart rate, perspiration, sleep patterns, or diet, of the human subject.

In Example 18, the subject matter of any one or more of Examples 13-17 optionally include the electronic operations further including: wirelessly communicating with the sensor device using a wireless communication protocol; wherein the electronic operations of receiving the sensor data from the sensor device are performed using the wireless communication protocol.

In Example 19, the subject matter of any one or more of Examples 11-18 optionally include the electronic operations further including: communicating the sensor data to a remote data processing system; and receiving an indication of the trigger of the pain condition from the remote data processing system, in response to communication of the sensor data and analysis by the remote data processing system; wherein the remote data processing system applies a trigger identification model to identify the trigger from the sensor data.

In Example 20, the subject matter of any one or more of Examples 11-19 optionally include wherein the plurality of ambient stimuli relates to at least two of light, sound, or odor, and wherein the sensor data indicates a level, a pattern, and a type for respective stimulus of the plurality of ambient stimuli.

Example 21 is at least one machine readable medium including instructions, which when executed by a computing system, cause the computing system to perform any of the methods of Examples 11-20.

Example 22 is an apparatus comprising means for performing any of the methods of Examples 11-20.

Example 23 is an apparatus, comprising: means for receiving sensor data from a sensor device worn by a human subject, wherein the sensor data indicates respective conditions of a plurality of ambient stimuli occurring in proximity to the human subject; means for receiving input from the human subject, wherein the input indicates a pain condition of the human subject; means for processing the sensor data and the received input with a user-trained model to identify a trigger of the pain condition, wherein the trigger of the pain condition corresponds to a particular stimulus of the plurality of ambient stimuli; and means for generating output to the human subject, wherein the output indicates the trigger of the pain condition.

In Example 24, the subject matter of Example 23 optionally includes wherein the pain condition of the human subject is a migraine headache, wherein the trigger of the pain condition relates to an environmental characteristic of the particular stimulus, and wherein the generated output includes an identification of the environmental characteristic of the particular stimulus as the trigger of the migraine headache.

In Example 25, the subject matter of Example 24 optionally includes wherein the means for processing the sensor data and the received input is used to identify the trigger of the migraine headache from a plurality of potential trigger conditions indicated in the sensor data, wherein the plurality of potential triggers are identified from sensor data produced for the plurality of ambient stimuli, and wherein the trigger is identified based on a degree of correlation of the trigger in a past occurrence of a migraine headache with the human subject.

In Example 26, the subject matter of any one or more of Examples 24-25 optionally include wherein the received input further includes feedback of a negative weighting of other stimuli characteristics from the plurality of ambient stimuli, and wherein, based on the negative weighting, the other stimuli characteristics from the plurality of ambient stimuli are identified as not being the trigger of the migraine headache.

In Example 27, the subject matter of any one or more of Examples 25-26 optionally include means for receiving additional input from the human subject, wherein the input and the input is received in a graphical user interface that is provided on a display of the computing device, wherein the input indicates a phase of the migraine headache, and wherein the additional input indicates a time of occurrence of a particular phase of the migraine headache; and means for determining a time period corresponding to the particular phase of the migraine headache; wherein the trigger of the migraine headache is identified based on sensor data captured in the time period corresponding to the particular phase of the migraine headache.

In Example 28, the subject matter of Example 27 optionally includes means for wirelessly communicating with a physiological sensor device; wherein the physiological sensor device includes a physiological sensor to produce physiologic sensor data captured from physiologic characteristics of the human subject; and wherein the trigger of the migraine headache is further identified based on the physiologic sensor data.

In Example 29, the subject matter of Example 28 optionally includes wherein the physiologic sensor data indicates one or more of: heart rate, perspiration, sleep patterns, or diet, of the human subject.

In Example 30, the subject matter of any one or more of Examples 25-29 optionally include means for wirelessly communicating with the sensor device using a wireless communication protocol; wherein the electronic operations of receiving the sensor data from the sensor device are performed using the wireless communication protocol.

In Example 31, the subject matter of any one or more of Examples 23-30 optionally include means for communicating the sensor data to a remote data processing system; and means for receiving an indication of the trigger of the pain condition from the remote data processing system, in response to communication of the sensor data and analysis by the remote data processing system; wherein the remote data processing system applies a trigger identification model to identify the trigger from the sensor data.

In Example 32, the subject matter of any one or more of Examples 23-31 optionally include wherein the plurality of ambient stimuli relates to at least two of light, sound, or odor, and wherein the sensor data indicates a level, a pattern, and a type for respective stimulus of the plurality of ambient stimuli.

Example 33 is a migraine trigger detection device for use by a human subject, the migraine trigger detection device comprising: an ambient stimuli sensor; sensor circuitry to collect sensor data from the ambient stimuli sensor; a wireless transceiver to communicate the sensor data to a computing device, and receive an indication of a migraine headache condition from processing with a user-trained model, wherein the sensor data indicates respective conditions of a plurality of ambient stimuli occurring in proximity to the human subject; and a processing logic component to indicate a trigger of the migraine headache condition, wherein the trigger of the migraine headache condition corresponds to a level, a pattern, and a type of a particular stimulus of the plurality of ambient stimuli.

In Example 34, the subject matter of Example 33 optionally includes wherein the migraine trigger detection device is integrated into a set of a wearable eyeglasses.

In Example 35, the subject matter of Example 34 optionally includes wherein the wearable eyeglasses include a display screen, wherein the display screen provides a user interface that outputs an indication of the trigger of the migraine headache condition.

In Example 36, the subject matter of any one or more of Examples 33-35 optionally include wherein the ambient stimuli sensor includes at least one of: a light sensing component; an odorous compound sensing component; or a sound sensing component.

In Example 37, the subject matter of any one or more of Examples 33-36 optionally include wherein the computing device performs operations to process the sensor data, receive input that indicates a condition and a timing of the migraine headache condition, and communicate the input that indicates the condition and the timing of the migraine headache condition to the migraine trigger detection device.

Example 38 is a method, comprising electronic operations executed in processing circuitry of a migraine trigger detection device, wherein the electronic operations include: collecting sensor data from the ambient stimuli sensor; communicating the sensor data to a computing device and receiving an indication of a migraine headache condition from the computing device, wherein the computing device performs processing of the sensor data with a user-trained model, and wherein the sensor data indicates respective conditions of a plurality of ambient stimuli occurring in proximity to the human subject; and indicating a trigger of the migraine headache condition based on an indication of the trigger received from the computing receive, wherein the trigger of the migraine headache condition corresponds to a level, a pattern, and a type of a particular stimulus of the plurality of ambient stimuli.

In Example 39, the subject matter of Example 38 optionally includes wherein the migraine trigger detection device is integrated into a set of a wearable eyeglasses.

In Example 40, the subject matter of Example 39 optionally includes wherein the wearable eyeglasses include a display screen, wherein the display screen outputs a user interface that outputs an indication of the trigger of the migraine headache condition.

In Example 41, the subject matter of any one or more of Examples 38-40 optionally include wherein the ambient stimuli sensor includes at least one of: a light sensing component; an odorous compound sensing component; or a sound sensing component.

In Example 42, the subject matter of any one or more of Examples 38-41 optionally include wherein the computing device performs operations to process the sensor data, receive input that indicates a condition and a timing of the migraine headache condition, and communicate the input that indicates the condition and the timing of the migraine headache condition to the migraine trigger detection device.

Example 43 is at least one machine readable medium including instructions, which when executed by a computing system, cause the computing system to perform any of the methods of Examples 38-42.

Example 44 is an apparatus comprising means for performing any of the methods of Examples 38-42.

Example 45 is an apparatus, comprising: means for sensing ambient stimuli; means for collecting sensor data from the means for sensing ambient stimuli; means for communicating the sensor data to a computing device and receiving an indication of a migraine headache condition from the computing device, wherein the computing device performs processing of the sensor data with a user-trained model, and wherein the sensor data indicates respective conditions of a plurality of ambient stimuli occurring in proximity to the human subject; and means for indicating a trigger of the migraine headache condition based on an indication of the trigger received from the computing receive, wherein the trigger of the migraine headache condition corresponds to a level, a pattern, and a type of a particular stimulus of the plurality of ambient stimuli.

In Example 46, the subject matter of Example 45 optionally includes means for outputting a user interface to output an indication of the trigger of the migraine headache condition.

In Example 47, the subject matter of any one or more of Examples 45-46 optionally include means for processing the sensor data and receiving input that indicates a condition and a timing of the migraine headache condition, wherein the input indicates the condition and the timing of the migraine headache condition.

Example 48 is a system, comprising: a migraine trigger detection device for use by a human subject, the migraine trigger detection device including: an ambient stimuli sensor; sensor circuitry to collect sensor data from the ambient stimuli sensor; and a wireless transceiver to communicate the sensor data; and a computing device for use by the human subject, the computing device including: processing and memory circuitry; a wireless transceiver to receive the sensor data from the migraine trigger detection device; and a trigger data processing component implemented with the processing circuitry and the memory circuitry, wherein the trigger data processing component implements: a trigger data aggregator to identify respective conditions of a plurality of ambient stimuli, from the sensor data, that occur in proximity to the human subject; pain input processing to evaluate input received from the human subject, wherein the input from the human subject indicates a migraine headache condition of the human subject; trigger window timing processing to identify a time interval relevant to the migraine headache condition of the human subject; and report generation processing to generate a trigger report to output to the human subject, wherein the trigger report indicates a stimuli trigger of the migraine headache condition.

In Example 49, the subject matter of Example 48 optionally includes wherein the trigger data processing component further implements: a trigger data correlation processing component to process the sensor data and the received input to identify the stimuli trigger of the migraine headache condition; wherein the stimuli trigger of the migraine headache condition corresponds to a particular stimulus of the plurality of ambient stimuli.

In Example 50, the subject matter of any one or more of Examples 48-49 optionally include wherein the computing device includes a display screen for an output of a graphical user interface, wherein the graphical user interface outputs the trigger report, and wherein the graphical user interface receives input corresponding to the time interval and phase selection interface.

In Example 51, the subject matter of any one or more of Examples 48-50 optionally include wherein the ambient stimuli sensor includes at least one of: a light sensing component; an odorous compound sensing component; or a sound sensing component.

In Example 52, the subject matter of any one or more of Examples 48-51 optionally include a wearable device, comprising: a sensor to collect physiological data; sensor circuitry to collect and process the physiological data from the sensor; and a wireless transceiver to communicate with the computing device; wherein the wearable device is in communication with the computing device to collect additional physiological data of the human subject; and wherein identification of the stimuli trigger is performed based on a combination of the sensor data and the physiological data.

In Example 53, the subject matter of any one or more of Examples 48-52 optionally include a remote processing computing system in communication with the computing device, the remote processing computing system to process the sensor data and the received input to identify a trigger of the migraine headache condition, wherein the trigger of the migraine headache condition corresponds to a particular stimulus of the plurality of ambient stimuli.

In the above Detailed Description, various features may be grouped together to streamline the disclosure. However, the claims may not set forth every feature disclosed herein as embodiments may feature a subset of said features. Further, embodiments may include fewer features than those disclosed in a particular example. Thus, the following claims are hereby incorporated into the Detailed Description, with a claim standing on its own as a separate embodiment.

What is claimed is:

1. A computing device, comprising processing circuitry to:
receive sensor data from a sensor device worn by a human subject, wherein the sensor data indicates respective conditions of a plurality of ambient stimuli occurring in proximity to the human subject;
receive input from the human subject, wherein the input indicates a pain condition of the human subject;
process the sensor data and the received input with a user-trained model to identify a trigger of the pain condition, wherein the trigger of the pain condition corresponds to a particular stimulus of the plurality of ambient stimuli; and
generate output to the human subject, wherein the output indicates the trigger of the pain condition.

2. The computing device of claim 1,
wherein the pain condition of the human subject is a migraine headache,
wherein the trigger of the pain condition relates to an environmental characteristic of the particular stimulus, and
wherein the generated output includes an identification of the environmental characteristic of the particular stimulus as the trigger of the migraine headache.

3. The computing device of claim 2,
wherein the operations that process the sensor data and the received input are used to identify the trigger of the migraine headache from a plurality of potential trigger conditions indicated in the sensor data,
wherein the plurality of potential triggers are identified from sensor data produced for the plurality of ambient stimuli, and
wherein the trigger is identified based on a degree of correlation of the trigger in a past occurrence of a migraine headache with the human subject.

4. The computing device of claim 2,
wherein the received input further includes feedback from the user of a negative weighting of other stimuli characteristics from the plurality of ambient stimuli, and
wherein, based on the negative weighting, the other stimuli characteristics from the plurality of ambient stimuli are identified as not being the trigger of the migraine headache.

5. The computing device of claim 3, the processing circuitry further to:
receive additional input from the human subject, wherein the input and the input is received in a graphical user interface that is provided on a display of the computing device, wherein the input indicates a phase of the migraine headache, and wherein the additional input indicates a time of occurrence of a particular phase of the migraine headache; and
determine a time period corresponding to the particular phase of the migraine headache;
wherein the trigger of the migraine headache is identified based on sensor data captured in the time period corresponding to the particular phase of the migraine headache.

6. The computing device of claim 5, further comprising:
a wireless transceiver to wirelessly communicate with a physiological sensor device;
wherein the physiological sensor device includes a physiological sensor to produce physiologic sensor data captured from physiologic characteristics of the human subject; and
wherein the trigger of the migraine headache is further identified based on the physiologic sensor data.

7. The computing device of claim 6,
wherein the physiologic sensor data indicates one or more of: heart rate, perspiration, sleep patterns, or diet, of the human subject.

8. The computing device of claim 3, further comprising:
a wireless transceiver to wirelessly communicate with the sensor device using a wireless communication protocol;
wherein the operations to receive the sensor data from the sensor device are performed using the wireless communication protocol.

9. The computing device of claim 1, the processing circuitry further to:
communicate the sensor data to a remote data processing system; and
receive an indication of the trigger of the pain condition from the remote data processing system, in response to communication of the sensor data and analysis by the remote data processing system;
wherein the remote data processing system applies a trigger identification model to identify the trigger from the sensor data.

10. The computing device of claim 1,
wherein the plurality of ambient stimuli relates to at least two of light, sound, or odor, and
wherein the sensor data indicates a level, a pattern, and a type for respective stimulus of the plurality of ambient stimuli.

11. At least one machine readable storage medium, comprising a plurality of instructions that, responsive to being executed with processor circuitry of a computing device, cause the computing device to perform electronic operations that:
receive sensor data from a sensor device worn by a human subject, wherein the sensor data indicates respective conditions of a plurality of ambient stimuli occurring in proximity to the human subject;
receive input from the human subject, wherein the input indicates a pain condition of the human subject;
process the sensor data and the received input with a user-trained model to identify a trigger of the pain condition, wherein the trigger of the pain condition corresponds to a particular stimulus of the plurality of ambient stimuli; and
generate output to the human subject, wherein the output indicates the trigger of the pain condition.

12. The machine readable storage medium of claim 11,
wherein the pain condition of the human subject is a migraine headache,
wherein the trigger of the pain condition relates to an environmental characteristic of the particular stimulus, and
wherein the generated output includes an identification of the environmental characteristic of the particular stimulus as the trigger of the migraine headache.

13. The machine readable storage medium of claim 12,
wherein the electronic operations that process the sensor data and the received input are used to identify the trigger of the migraine headache from a plurality of potential trigger conditions indicated in the sensor data,
wherein the plurality of potential triggers are identified from sensor data produced for the plurality of ambient stimuli, and
wherein the trigger is identified based on a degree of correlation of the trigger in a past occurrence of a migraine headache with the human subject.

14. The machine readable storage medium of claim 12,
wherein the received input further includes feedback of a negative weighting of other stimuli characteristics from the plurality of ambient stimuli, and
wherein, based on the negative weighting, the other stimuli characteristics from the plurality of ambient stimuli are identified as not being the trigger of the migraine headache.

15. The machine readable storage medium of claim 13, wherein the electronic operations further:
receive additional input from the human subject, wherein the input and the input is received in a graphical user interface that is provided on a display of the computing device, wherein the input indicates a phase of the migraine headache, and wherein the additional input indicates a time of occurrence of a particular phase of the migraine headache; and
determine a time period corresponding to the particular phase of the migraine headache;
wherein the trigger of the migraine headache is identified based on sensor data captured in the time period corresponding to the particular phase of the migraine headache.

16. The machine readable storage medium of claim 15, wherein the electronic operations further:
wirelessly communicate with a physiological sensor device;
wherein the physiological sensor device includes a physiological sensor to produce physiologic sensor data captured from physiologic characteristics of the human subject; and
wherein the trigger of the migraine headache is further identified based on the physiologic sensor data.

17. The machine readable storage medium of claim 16,
wherein the physiologic sensor data indicates one or more of: heart rate, perspiration, sleep patterns, or diet, of the human subject.

18. The machine readable storage medium of claim 13, wherein the electronic operations further:
wirelessly communicate with the sensor device using a wireless communication protocol;
wherein the electronic operations that receive the sensor data from the sensor device are performed using the wireless communication protocol.

19. The machine readable storage medium of claim 11, wherein the electronic operations further:
communicate the sensor data to a remote data processing system; and
receive an indication of the trigger of the pain condition from the remote data processing system, in response to communication of the sensor data and analysis by the remote data processing system;
wherein the remote data processing system applies a trigger identification model to identify the trigger from the sensor data.

20. The machine readable storage medium of claim 11,
wherein the plurality of ambient stimuli relates to at least two of light, sound, or odor, and
wherein the sensor data indicates a level, a pattern, and a type for respective stimulus of the plurality of ambient stimuli.

21. A migraine trigger detection device for use by a human subject, the migraine trigger detection device comprising:
an ambient stimuli sensor;
sensor circuitry to collect sensor data from the ambient stimuli sensor;
a wireless transceiver to communicate the sensor data to a computing device, and receive an indication of a migraine headache condition from processing with a user-trained model, wherein the sensor data indicates respective conditions of a plurality of ambient stimuli occurring in proximity to the human subject; and
a processing logic component to indicate a trigger of the migraine headache condition, wherein the trigger of the migraine headache condition corresponds to a level, a pattern, and a type of a particular stimulus of the plurality of ambient stimuli.

22. The migraine trigger detection device of claim 21,
wherein the migraine trigger detection device is integrated into a set of a wearable eyeglasses.

23. The migraine trigger detection device of claim 22,
wherein the wearable eyeglasses include a display screen,
wherein the display screen provides a user interface that outputs an indication of the trigger of the migraine headache condition.

24. The migraine trigger detection device of claim 21, wherein the ambient stimuli sensor includes at least one of:
a light sensing component;
an odorous compound sensing component; or
a sound sensing component.

25. The migraine trigger detection device of claim 21,
wherein the computing device performs operations to process the sensor data, receive input that indicates a condition and a timing of the migraine headache condition, and communicate the input that indicates the condition and the timing of the migraine headache condition to the migraine trigger detection device.
